(19) United States
(12) Patent Application Publication
(10) Pub. No.: US 2022/0096795 A1
(43) Pub. Date: Mar. 31, 2022

(54) GUIDE EXTENSION CATHETER

(71) Applicant: Teleflex Life Sciences Limited, Valletta (MT)

(72) Inventor: Joshua Brenizer, Oak Grove, MN (US)

(73) Assignee: Teleflex Life Sciences Limited, Valletta (MT)

(21) Appl. No.: 17/425,068

(22) PCT Filed: Dec. 12, 2019
(86) PCT No.: PCT/US19/65844

§ 371 Date: Jul. 22, 2021

Related U.S. Application Data
(60) Provisional application No. 62/807,613, filed on Feb. 19, 2019.

(51) Int. Cl. A61M 25/01; A61M 25/02; A61M 25/06; A61M 25/09

(52) U.S. Cl. CPC A61M 25/0102; A61M 25/02; A61M 25/0662; A61M 2025/0293; A61M 25/0138; A61M 2025/0687; A61M 25/09

(57) ABSTRACT

Guide extension catheters and related methods are disclosed. A guide extension catheter can comprise, among other things, an elongate tube member and a push member. The push member can be operably coupled to the tube member at an eccentric coupling position and can extend proximally therefrom for slidably positioning the elongate tube member over and partially beyond a distal end of the guide catheter. The inner diameter of the elongate tube member may be adjustable, such that it constricts when not bound along its inner surface by the guide catheter. The elongate tube member can, for example, include a longitudinal slit configured to expand and contract to accommodate insertion and removal of the guide catheter, respectively.

### CLAIM OF PRIORITY

Benefit of priority is hereby claimed to U.S. provisional patent application bearing Ser. No. 62/807,613, entitled “GUIDE EXTENSION CATHETER” and filed on Feb. 19, 2019, which is herein incorporated by reference in its entirety.

### TECHNICAL FIELD

The subject matter of this patent document relates to the field of medical devices. More particularly, but not by way of limitation, the subject matter relates to guide extension catheters for use with guide catheters.

### BACKGROUND

Interventional cardiology procedures often involve inserting guidewires or other instruments through catheters into coronary arteries that branch off from the aorta. In coronary artery disease, the coronary artery may be narrowed or occluded by atherosclerotic plaques or other lesions. These lesions may totally obstruct the lumen of the artery or may dramatically narrow the lumen of the artery. A narrowing is referred to as a stenosis. In order to diagnose and treat obstructive coronary artery disease it is commonly necessary to pass a guidewire or other instruments through and beyond the occlusion or stenosis of the coronary artery.

To treat a stenosis, a guide catheter can be inserted through the aorta and into or adjacent the ostium of the coronary artery. This is sometimes accomplished with the aid of the guidewire. The guide catheter is typically seated into or adjacent the opening or ostium of the artery to be treated and the guidewire or other instrument is passed through the lumen of the guide catheter and inserted into the artery beyond the occlusion or stenosis. Crossing tough lesions or tortuous anatomy can create enough backward force to dislodge the guide catheter from the ostium of the artery being treated. This can make it difficult or impossible for the interventional cardiologist to treat certain forms of coronary artery disease.

A coaxial guide catheter can be used in conjunction with a standard guide catheter to provide additional backup support. The coaxial guide catheter can be passed through the standard guide catheter until its distal end extends beyond the distal end of the standard guide catheter, thereby positioning the distal end of the coaxial guide catheter further within the branch artery harboring the stenosis. Coaxial guide catheters may thus be referred to as guide extension catheters.

While preexisting guide extension catheters may provide increased backup support, the structure of such catheters may interfere with the insertion of interventional devices and may be limited with respect to size. Accordingly, new devices and associated techniques capable of minimizing device interactions and accessing differently sized vessels are desired.

### OVERVIEW

The present inventor recognizes that there is a need to provide guide extension catheters that are compatible with guide catheters for performing interventional procedures in challenging anatomy, e.g., narrow blood vessels harboring robust occlusions. The present inventor also recognizes that there is a need to reduce structural interactions between guide extension catheters and interventional devices inserted therethrough. The present inventor further recognizes that guide extension catheters having various diameters are also needed to perform interventional procedures in differently sized vessels. A guide extension catheter can be configured for delivery over a standard guide catheter to access discrete regions of coronary or peripheral vasculature and to facilitate accurate placement of interventional devices without interfering with the passage of such devices through the guide catheter. A guide extension catheter can further include distal tubing having a variable cross-sectional diameter.

Guide extension catheters and related methods are disclosed in this patent document. A guide extension catheter can comprise an elongate tube member (also referred to as guide extension tubing) and a push member (also referred to as a substantially rigid portion, push rod, or push wire). The push member, which may not have a lumen large enough to allow passage of interventional cardiology devices in some examples, can be eccentrically coupled relative to the tube member for slidably positioning the tube member around and partially beyond a distal end of a guide catheter into a vessel ostium of interest.

These and other embodiments and features of the present guide extension catheters and related methods will be set forth, at least in part, in the following Detailed Description. This Overview is intended to provide non-limiting embodiments of the present subject matter; it is not intended to provide an exclusive or exhaustive explanation of the disclosed embodiments. The Detailed Description below is included to provide further information about the present guide extension catheters and methods.

### BRIEF DESCRIPTION OF THE DRAWINGS

In the drawings, like numerals can be used to describe similar features and components throughout the several views. The drawings illustrate generally, by way of example, but not by way of limitation, various embodiments discussed in this patent document.

FIG. 1 illustrates a plan view of a guide catheter advanced through an aorta to an ostium of a coronary vessel.

FIG. 2 illustrates a plan view of a guide extension catheter, as constructed in accordance with at least one embodiment, used in conjunction with a guide catheter for the delivery of an interventional device into an occluded vessel for treatment.

FIG. 3A illustrates a side view of a guide extension catheter, as constructed in accordance with at least one embodiment, showing an elongate tube member surrounding a guide catheter.

FIG. 3B illustrates a side view of the guide extension catheter of FIG. 3A, showing a portion of the elongate tube member extended beyond a distal end of the guide catheter.

FIG. 4 illustrates a cross-sectional view of a push member, as constructed in accordance with at least one embodiment, surrounding a guide catheter and within an introducer sheath.

FIG. 5 illustrates a cross-sectional view of the push member of FIG. 4, as constructed in accordance with at least one embodiment, showing an elongate tube member surrounding the guide catheter.

FIG. 6 illustrates a perspective view of a guide extension catheter, as constructed in accordance with at least one embodiment.

FIG. 7 illustrates a perspective view of another guide extension catheter, as constructed in accordance with at least one embodiment.

FIG. 8A illustrates a perspective view of another guide extension catheter surrounding a portion of a guide catheter, as constructed in accordance with at least one embodiment.

FIG. 8B illustrates a cross-sectional view of a the guide extension catheter of FIG. 8A.

FIG. 9 illustrates a cross-sectional view of another guide extension catheter, as constructed in accordance with at least one embodiment.

FIG. 10 illustrates a perspective view of another guide extension catheter surrounding a portion of a guide catheter, showing a tubular push member defining a longitudinal slit.

FIG. 11 illustrates a perspective view of another guide extension catheter surrounding a portion of a guide catheter, showing a tubular push member configured to constrict.

FIG. 12A illustrates a perspective view of another guide extension catheter surrounding a portion of a guide catheter, showing a push member configured to snap or clip ono the guide catheter.

FIG. 12B illustrates a cross-sectional view of the guide extension catheter of FIG. 12A.

FIG. 13A illustrates a perspective view of a reinforcement member included in a guide extension catheter in accordance with at least one embodiment.

FIG. 13B illustrates a perspective view of another reinforcement member included in a guide extension catheter in accordance with at least one embodiment.

The drawings are not necessarily to scale. Certain features and components may be shown exaggerated in scale or in schematic form, and some details may not be shown in the interest of clarity and conciseness.

### DETAILED DESCRIPTION

This patent document discloses guide extension catheters to be placed over guide catheters for providing support and guidance in a vessel when percutaneously advancing interventional devices, such as balloon catheters, stents, or stent catheters. A guide extension catheter is configured to be passed over a guide catheter so that its distal end portion can be extended beyond a distal end of the guide catheter and into the desired vessel while its proximal or intermediate portions may remain around an outer surface of the guide catheter.

It is believed that the present guide extension catheters will find great utility by interventional cardiologists performing percutaneous transluminal coronary interventions. Although the remainder of this patent document generally discusses and illustrates such uses, it should be understood that the guide extension catheters can also be used for treating other non-coronary diseased vessels or other hollow structures (e.g., biliary tract, ureter, etc.) throughout a patient's body where interventional devices are or can be employed.

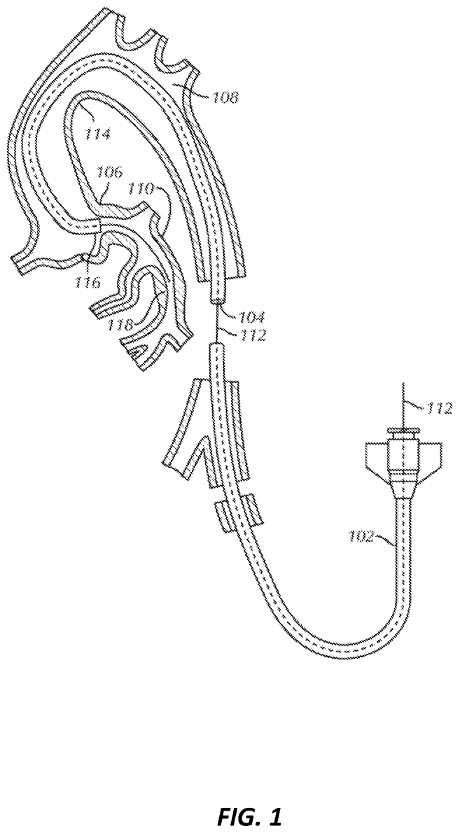

Minimally invasive cardiac interventions are utilized throughout the world and often include the use of a guidewire 112 and a guide catheter 102, as illustrated in FIG. 1. The guidewire 112 can comprise an elongate, small-diameter member designed to navigate vessels to reach a diseased site or vessel segment of interest. Guidewires can come in various configurations, including solid steel or nitinol core wires and/or solid core wire wrapped in a smaller wire coil or braid, for example. The guide catheter 102 can comprise an elongate tube member defining a main lumen 104 along its length. The guide catheter 102 can be formed of polyurethane, for example, and can be shaped to facilitate its advancement to a coronary ostium 106 (or other region of interest within a patient's body). Any sized guide catheter 102, such as a 6F, 7F, 8F guide catheter, where F is an abbreviation for the French catheter scale (a unit to measure catheter diameter (1F=⅓ mm)), can be inserted at a femoral or radial artery and advanced through an aorta 108 to a position adjacent to the ostium 106 of a coronary artery 110.

In a typical procedure, the guidewire 112 and guide catheter 102 can be advanced through the arch 114 of the aorta 108 to the ostium 106. The guidewire 112 may then be advanced beyond the ostium 106 and into the coronary artery 110. The diameter and rigidity of the guide catheter's distal end 116, however, may not permit the device to be advanced beyond the ostium 106 and into the coronary artery 110.

Maintaining the position of the guide catheter's distal end 116 at the ostium 106 can facilitate the guidewire 112 or other interventional device successfully reaching the diseased site (e.g., a stenotic lesion 118) through its further distal advancement. With the guide catheter 102 in position, force can be applied to the guidewire's proximal end to push the guidewire 112 to and beyond the lesion 118, and a treating catheter (optionally including a balloon or stent) can be passed over the guidewire 112 to treat the site. The application of force to the guidewire 112 or the treating catheter can sometimes cause the guide catheter 102 to dislodge from the ostium 106 of the coronary artery 110, and, in such instances, the guidewire or treating catheter must be further distally advanced independently of the guide catheter's alignment and support to reach the lesion 118. This can occur in the case of a tough stenotic lesion 118 or tortuous anatomy, where it is often difficult to pass the guidewire 112 or the treating catheter to and beyond the lesion. A heart's intrinsic beat can also cause the guide catheter's distal end 116 to lose its positioning or otherwise be shifted so that it no longer is positioned to align and support the guidewire 112 or the treating catheter into the portion of the coronary artery 110 including the lesion 118.

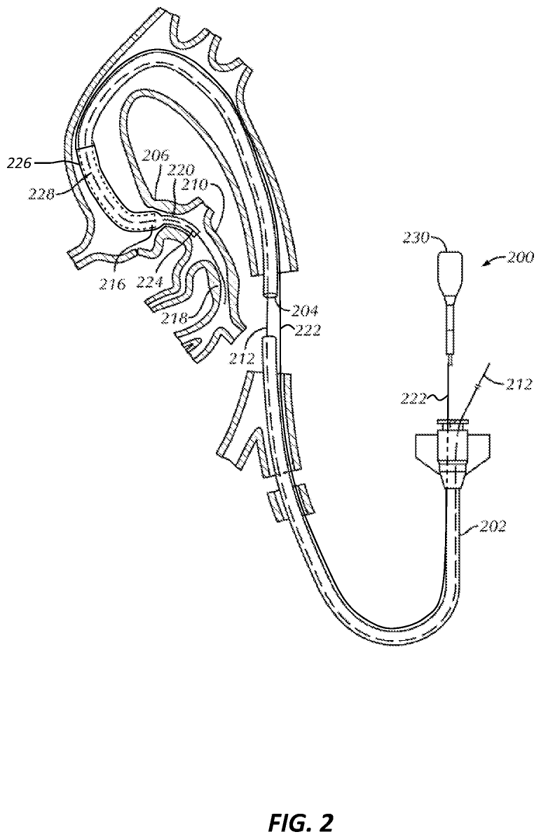

As illustrated in FIG. 2, the present guide extension catheter 200 can improve access to a coronary artery 210 and a stenotic lesion 218. The guide extension catheter 200 can include a relatively flexible elongate tube member 220 and a push member 222 having a collective length that is greater than a length of a guide catheter 202 (e.g., 130 cm-175 cm, or greater). In embodiments, the combined length of the push member 222 and elongate tube member 220 can be limited such that upon full distal extension of the guide extension catheter 200, the elongate tube member 220 does not fully extend beyond a distal end 216 of the guide catheter 202. By sizing the guide extension catheter 200 in this manner, at least a proximal portion of the elongate tube member 220 may always surround a portion of the guide catheter 200, thereby not interfering with the extension of interventional devices beyond the guide catheter's distal end 216. In addition or alternatively, the guide extension catheter 200 may include one or more structures configured to limit its distal extension, such as a proximal, outwardly-fanning funnel having a cross-sectional diameter larger than that of the guide catheter 202 and/or an introducer sheath. An outer diameter of the tube member 220 can be sized to permit insertion of its distal end portion 224 into a coronary artery or its branches containing the lesion 218, thereby providing alignment and support for an interventional device (e.g., a treating catheter) beyond the distal end 216 of the guide catheter 202 to the lesion and beyond. The extension of the tube member 220 into a smaller-sized artery or branch also serves to maintain the position of the guide catheter 202 at an artery's ostium 206 during an operation.

The operating physician can advance the distal end portion 224 of the tube member 220 over a guidewire 212 and the guide catheter 202 until it extends beyond the guide catheter's distal end 216 into the coronary artery 210 by applying a longitudinal force to the push member 222. As shown, at least a distal portion of the tube member 220 may comprise a structure and/or material configured to constrict, such that all or a portion of the tube member 220 decreases in diameter after its extension beyond the guide catheter's distal end 216. A proximal end portion 226 of the tube member 220 can remain around the guide catheter 202. The physician can subsequently deliver a treating catheter over the guidewire 212, through a main lumen 204 of the guide catheter 202 and a lumen 228 of the tube member 220 until the working portion of the treating catheter is located beyond the distal end portion 224 of the tube member. The operating physician can then treat the lesion 218 using standard techniques with added backup support on the guide catheter 202, thereby providing an extra ability to push and advance the treating catheter.

In general, the lumen 228, and hence the tube member 220, can be sized and shaped to pass one or more interventional devices such as the guidewire 212, guide catheter 202, and treating catheter therethrough. The cross-sectional shape of the lumen 228 can be similar to the cross-sectional shape of the guide catheter's main lumen 204. For instance, in some examples, the cross-sectional shape of the lumen 228 can be generally uniform along its length. In other examples, the cross-sectional diameter may vary along the length of the tube member 220.

The outer diameter of the tube member 220 can assume minimum cross-sectional dimensions that allow the tube member 220 to coaxially slide over the guide catheter 202. In other embodiments, the outer cross-sectional dimensions of the tube member 220 can be greater than the allowable minimum. For example, with a 6F guide catheter, the tube member 220 can have about a 7F, 8F, 9F or greater diameter, or any diameter therebetween. In some embodiments, a diameter of the lumen 228 of the tube member 220 may not be more than about one French size larger than an outer diameter of the guide catheter 202. In one embodiment, the guide extension catheter 200 can be made in at least three sizes corresponding to the internal capacity of 8F, 7F, and 6F guide catheters that are commonly used in interventional cardiology procedures. The difference in size between the inner diameter of the tube member 220 and the outer diameter of the guide catheter 202 may vary. For instance, the gap in cross-sectional diameter between the outer diameter of the guide catheter and the inner diameter of the tube member 220 may be less than and/or about 0.001 in., 0.002 in., 0.003 in., 0.004 in., or 0.005 in., or any distance therebetween. In specific embodiments, the cross-sectional diameter gap may range from about 0.002 to 0.003 in., or about 0.002 to 0.0035 in. The diameter gap between an inner diameter of the tube member 220 and outer diameter of the guide catheter 202 may also be generally continuous along a substantial portion of the length or a majority of the length of the tube member 220 in some embodiments. In various embodiments, a guide catheter 202 with any diameter may be used. The length of the tube member 220 can be substantially less than the length of the guide catheter 202; however, the tube member 220 can be designed with any length according to a desired application, such as about 6 to about 45 cm, about 10 to about 35 cm, about 14 to about 25 cm, or about 18 to about 20 cm.

The push member 222 can be attached to the proximal end portion 226 of the tube member 220 and can extend proximally from this attachment to a handle member 230 (also referred to as a manipulation member) accessible to an operating physician outside of a patient's body. The handle member 230 and push member 222 can allow the physician to position the tube member 220 between a first position, entirely surrounding the guide catheter 202, and the illustrated second position, in which the tube member's distal end 224 extends beyond that of the guide catheter 202 and into the coronary artery 210. The push member 222 can be rigid enough to allow the guide extension catheter 200 to be inserted over the guide catheter 202 upon receiving a pushing force from a physician via the handle member 230. In some examples, the push member 222 can be more rigid along its longitudinal axis than the tube member 220, and may comprise a rod, wire, or rail structure without a lumen through which interventional cardiology devices and the guide catheter 202 are insertable.

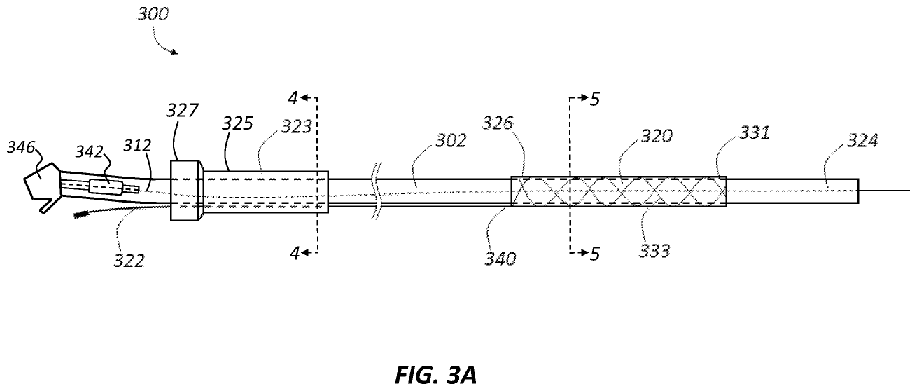

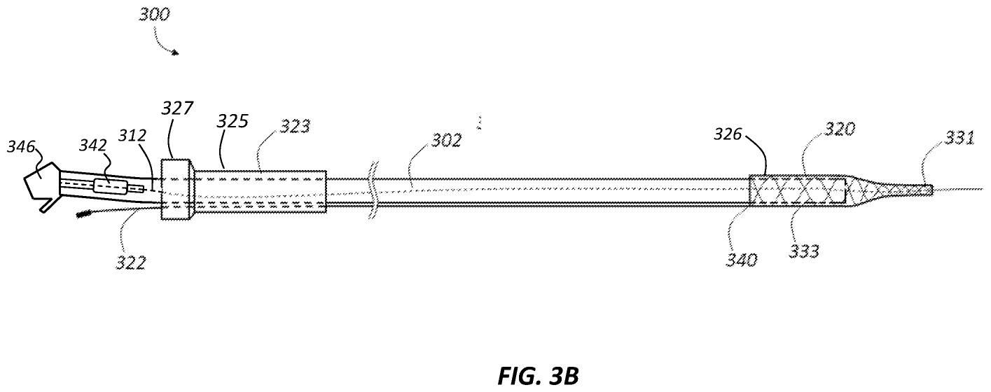

FIG. 3A illustrates a side view of an example guide extension catheter 300 in accordance with embodiments of the present disclosure. As shown, a guidewire 312 may extend through the guide extension catheter 300, which can include an elongate tube member 320 coupled at a proximal end 326 with a push member 322 at the push member's distal end 340. The tube member 320 can surround a portion of the guide catheter 302, and may be advanced distally over the guide catheter 302 until at least a distal portion of it surpasses the guide catheter's distal end 324. A hemostatic valve 346, which can be traversed using a preassembled kit, is coupled to a proximal end of the guide catheter 302.

An introducer sheath 323 is also included near the proximal end of both the guide catheter 302 and the push member 322. The introducer sheath 323 may generally include an elongate shaft 325 and a tubular entry port 327 configured to receive and facilitate entry and removal of the guide extension catheter 300 into a patient's vasculature. The tube member 320, or at least a distal portion 331 thereof, may include an elastic material or structure, such as the shape-memory braid 333 shown in FIG. 3, which may be comprised of nitinol in some examples.

The introducer sheath 323 may reduce lateral and axial movement of the guide catheter 302 and guide extension catheter 300 used during a vascular procedure relative to the blood vessel wall, thereby reducing or eliminating vessel spasm. The length of the introducer sheath 323 may vary depending on the depth of the targeted vessel relative to the skin, said depth ranging from about 1 cm to about 10 cm in some embodiments, or depths less or greater than this range. The introducer sheath 323 defines a lumen, the diameter of which may also vary, configured to receive various elongated vascular instruments. While the introducer sheath 323 shown in FIG. 3 is generally cylindrical, any cross-section may be used (and the cross-section can vary along the length), and the shape of the sheath may also vary, defining converging and/or asymmetrical tip portions, for example.

The size and configuration of the guidewire 312 may also vary. In some examples, a hollow guidewire defining a lumen may be used. In other embodiments, the guidewire 312 may be solid. The diameter of the guidewire may vary depending on the diameter of the vessel lumen and/or the diameter of the other instruments employed during the operation. Specific embodiments may include a guidewire having a diameter ranging from about a 0.01 in. to about 0.04 in., about 0.013 in. to about 0.038 in., about 0.015 in.

to about 0.02 in., or about 0.018 in. The length of the guidewire may also vary, including ranging from about 30 cm to about 270 cm, about 30 cm to about 80 cm, about 30 cm to about 70 cm, about 35 cm to about 45 cm, or about 55 cm to about 65 cm in various embodiments. In some examples, the guidewire may define a tapered tip portion at the distal end, which may also be curved to avoid perforation of the vessel wall. The guidewire 312 may comprise stainless steel in some embodiments. It is to be appreciated that the dimensions in this paragraph, and in this document, are exemplary only, and any suitable dimensions may be used.

With the guidewire 312 and the guide catheter 302 positioned as desired, the tube member 320 of the guide extension catheter 300 can be backloaded from its distal end portion 331 onto a proximal end of the guide catheter 302. As shown in FIG. 3B, a portion of the tube member 320 of the guide extension catheter 300 can then be advanced beyond a distal end 324 of the guide catheter 302 under fluoroscopy. When so arranged, portions of the tube member 320 can engage an ostium and extend within a portion of a coronary artery to help maintain the position of the guide catheter 302 as a treating catheter 342 is advanced.

The push member 322 can be rigid enough to urge the tube member 320 through the vasculature in response to receiving an axial force applied at a proximal end thereof, e.g., by a physician. The stiffness of the push member 322 may be uniform, or substantially uniform, along its length. In certain examples, the push member 322 can include a plurality of segments or portions having different stiffness and flexibility profiles to provide the guide extension catheter 300 with a desired combination of pushing force and vessel placement capabilities. In some embodiments, the push member 322 can be an elongated solid wire of constant or varying dimensions and can be made of a polymeric or metallic material, such as high tensile stainless steel (e.g., 304V, 304L or 316LV), mild steel, nickel-titanium allows, nickel-chromium-molybdenum alloys, nickel-copper alloys, nickel-tungsten alloys or tungsten alloys. The push member 322 can be coated with a hydrophilic, silicone or other friction-reducing material.

In some examples, the tube member 320 can be formed from an inner polymer layer, an outer polymer layer, and/or a reinforcement member (e.g., braid or coil) disposed between or adjacent to the polymer layers, for example as described in U.S. Pat. Nos. 8,048,032, 8,142,413, RE45,760, RE 45,380, RE45,776, and RE46,116, which are incorporated by reference in their entireties herein. According to such examples, the inner polymer layer can be composed of, or coated with, silicone, polytetrafluoroethylene (PTFE) or another lubricious material to provide a slippery surface for received interventional devices. The outer polymer layer can include one or more flexible materials, such as polyurethane, polyethylene or polyolefin of sequentially diminishing durometers along the tube member's length, and it can be coated with a friction-reducing material (e.g., a hydrophilic material) to facilitate insertion and trackability through vasculature and a guide catheter. The reinforcing braid or coil, in embodiments featuring a braid or coil, can be formed of stainless steel or a platinum alloy, for example, and can extend between the polymer layers along at least a portion of the tube member's length.

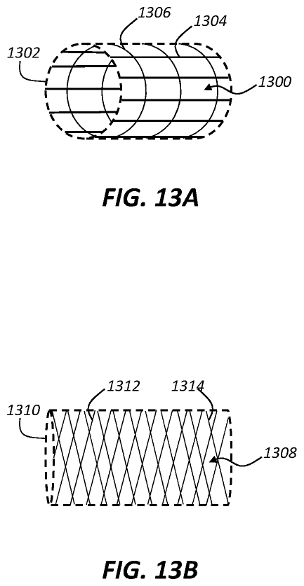

The optional reinforcement member disposed between the polymer layers of some elongate tube members 320 can be configured in multiple ways. For instance, the reinforcement member may lack a braid, coil or other distinct reinforcing structure, and may instead comprise one or more materials having greater stiffness than the remaining portions of the tube member 320. In addition or alternatively, embodiments of the reinforcement member can include different reinforcing structures, e.g., a rigid sleeve, elongate member, and/or bars or strips of rigid or semi-rigid material, as shown in FIGS. 13A and 13B. Additional components and/or materials configured to increase or decrease the rigidity of a portion of the tube member 320 are also contemplated. At least in part because the components of the reinforcement member may vary, methods of assembling the reinforcement member may also vary. For example, if the reinforcement member disposed between the polymer layers of the elongate member 320 includes a coil, various types of coils may be used, and in some examples, each coil can be coupled with other components of the tube member 320 in a distinct manner, which may depend on whether the cross-sectional diameter of the tube member is uniform or varied.

A proximal end portion 326 of the tube member 320 can be eccentrically coupled to a distal end portion 340 of the push member 322 at its periphery or circumference and can provide a smooth transition between the members in some examples. The arrangement or configuration of this coupling can vary. For example, the tube member 320 can include a side opening formed at a proximal end of its peripheral wall. In some examples, the push member 322 can be disposed within the opening. Inserting the push member 322 into the opening can result in a mechanical coupling between the members and additional or alternative bonds (e.g., adhesive bonds, thermal bonds, welds, brazes, etc.) can be utilized. The distal end portion 340 of the push member 322 can be flattened in some embodiments to provide a larger surface area to secure to the tube member 320. In addition or alternatively, coupling mechanisms facilitated by a third component (e.g., a metal or polymer skived (slanted) collar or concave track) bonded between or integrated with the proximal end portion 326 of the tube member 320 or the distal end portion 340 of the push member 322 are also contemplated, for example as described in U.S. patent application Ser. No. 15/581,176, which is incorporated by reference in its entirety herein. Metallic or polymeric structures forming the third component can become less stiff and more flexible in a proximal-to-distal direction, for instance, to provide a gradual flexibility transition between the more rigid push member 322 and the more flexible tube member 320.

Markers on the push member 322 or the tube member 320 can allow an operating physician to identify positioning of the guide extension catheter's components relative to patient anatomy, the guide catheter 302, and any interventional devices used during a procedure. For example, one or more depth markers can be printed on an outer surface of the push member 322 and can be positioned at predetermined lengths relative to a distal end of the tube member 320. One or more radiopaque marker bands can be positioned on the tube member 320. The marker bands can be composed of tungsten, platinum or an alloy thereof and can have a metallic band structure. Alternatively, for space conservation reasons, the marker bands can be formed by impregnating portions of the tube member 320 with a radiopaque filler material, such as barium sulfate, bismuth trioxide, bismuth carbonate, powdered tungsten, powdered tantalum or the like. A first marker band can be positioned slightly distal to a fully-round entrance of the tube member 320 and a second marker band can be positioned near the tube member's distal end, for example.

FIG. 3B illustrates another side view of the example guide extension catheter 300 shown in FIG. 3A, after distal extension of the tube member 320 via the push member 322. As shown, at least a distal portion 331 of the tube member 320 may be configured to shrink or constrict when no longer structurally bound from the interior by the guide catheter 302. Constriction of the tube member 320 may allow it to access narrow vessels without interfering with the passage of interventional devices through the guide catheter 302. Additional examples of the tube member 320 may not exhibit a variable cross-sectional diameter, such that even when extended beyond the distal end of the guide catheter 302, the tube member 320 may retain a generally constant or substantially constant diameter.

The decrease in cross-sectional diameter exhibited by examples of the tube member 320 configured to constrict may vary. For example, the diameter of the tube member 320 may decrease until its inner diameter matches or approximately matches the inner diameter of the guide catheter 302. In some embodiments, such as the one depicted in FIG. 3B, the diameter may decrease until the inner diameter of the tube member 320 is smaller than the inner diameter of the guide catheter 302. In various examples, the decreased inner diameter of the tube member 320 may be greater than 8F, or approximately 8F, 7F, 6F, 5F or less, or any diameter therebetween. The tube member 320 can be relatively flexible or elastic, such that its cross-sectional diameter increases again upon retraction over the guide catheter 302, or upon receipt and passage of one or more interventional devices therethrough.

FIG. 4 illustrates a cross-sectional view of an example push member 422 near its proximal end portion, such as along line 4-4 of FIG. 3A, within an introducer sheath 423 defining a proximal entry port 427. The push member 422 lies radially between the sheath 423 and a guide catheter 402, which defines a lumen 404 containing a guidewire 412. The push member 422 defines an oval cross-section in this particular example, but the cross-sectional shape and dimensions of the push member 422 may vary. For example, the push member 422 comprise an arcuate or flat, sheet-like cross-sectional shape, and rectangular, irregular, oval, oblong cross-sectional shapes are also within the scope of this disclosure. In operation, the guidewire 412, introducer sheath 423, and guide catheter 402 may all be introduced into a patient's vasculature prior to insertion of the guide extension catheter comprised of push member 422.

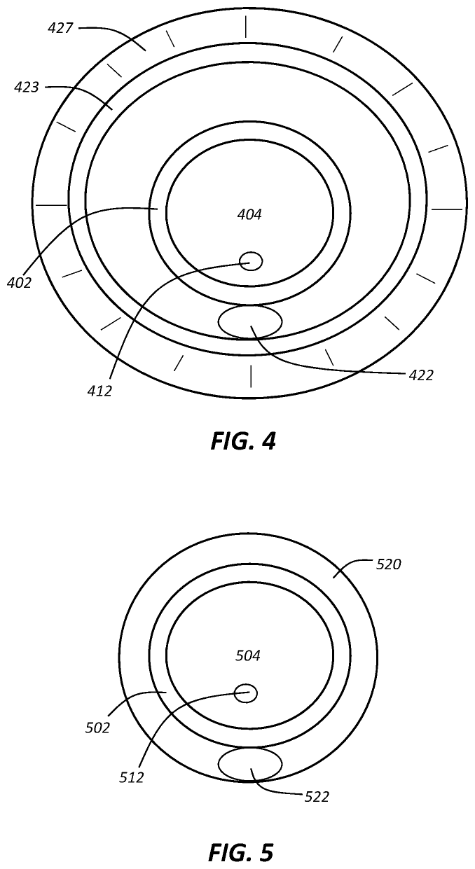

FIG. 5 illustrates a cross-sectional view of a distal end portion of an example push member 522, such as along line 5-5 of FIG. 3A. The push member 522 is coupled at a distal end with an elongate tube member 520, which surrounds a guide catheter 502, an inner lumen 504 of which includes a guidewire 512. The distal end of the push member 522, upon receiving an axial or longitudinal force by an operating physician, may contact and push the tube member 520 over the guide catheter 502. The relative thickness of each component shown in FIG. 5 may vary. For example, the tube member 520 may define a cross-sectional thickness that is greater than, less than, or generally equal to the thickness of the guide catheter 502.

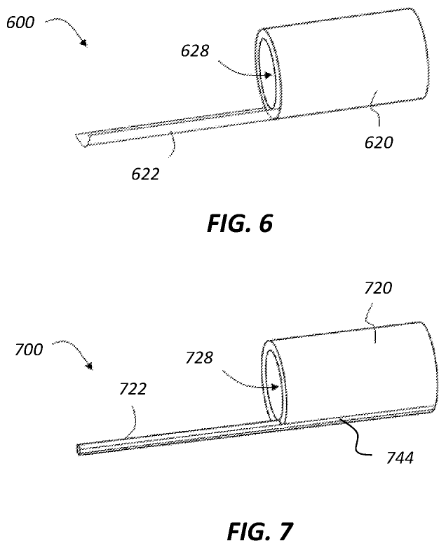

FIG. 6 illustrates a perspective view of a portion of a guide extension catheter 600 comprised of an elongate tube member 620 and a push member 622. The elongate tube member 620 can be generally cylindrical and may define a lumen 628 configured to receive a guide catheter. The configuration of the push member 622 may vary. In the example shown, the push member 622 defines an arcuate, concave track configured to act as a rail for a guide catheter, thereby facilitating relative movement between the guide catheter, the push member 622, and the elongate tube member 620. The arc length, flexibility and degree of curvature defined by the push member 622 can vary (as further described below), and may depend on the circumference of the guide catheter used with the guide extension catheter 600. For example, the arc length of the push member 622 may be greater to accommodate larger guide catheters, and vice versa.

FIG. 7 illustrates a perspective view of another guide extension catheter 700 comprised of an elongate tube member 720 defining a lumen 728 and attached to a push member 722. In this example, the push member 722 defines a cylindrical or rod-like shape, and along with the tube member 720, defines a longitudinal groove 744. The groove 744 may extend along the entire length of the tube member 720 and push member 722, or only a portion thereof. The groove 744 can receive and guide one or more additional interventional devices, e.g., guidewires, to the vessel target site. In some examples, the groove 744 may define a relatively smooth, concave surface, or straight opposing surfaces that meet at a point. Embodiments may include 2 or more grooves, for example on opposite sides of the guide extension catheter 700. The grooves 744 are not limited to any particular embodiment, and may be included in guide extension catheter 300 and/or 600, for instance.

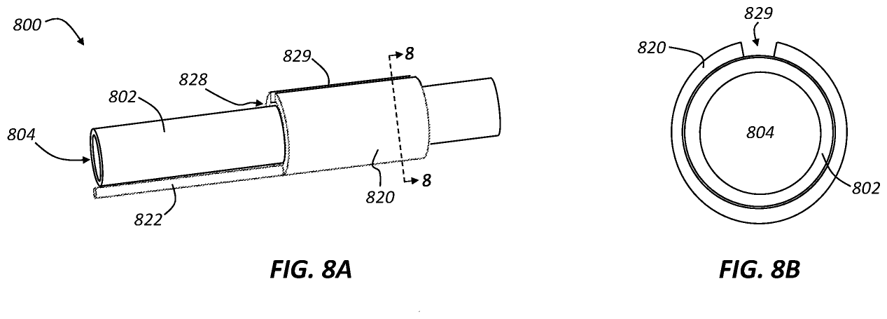

FIG. 8A illustrates a perspective view of another guide extension catheter 800 comprised of an elongate tube member 820 defining a lumen 828 and attached to a push member 822. A guide catheter 802 defining an inner lumen 804 is slidably coupled within the elongate tube member 820, extending through the tube member's lumen 828. The tube member 820 includes a longitudinal slit 829 that extends along its length. The slit 829 can have non-overlapping ends and can be formed to be resiliently closed, such that it can be forcibly peeled open to receive and/or remove the guide catheter 802. Embodiments may involve urging a proximal end of the guide catheter 802 into the elongate tube member 820 before, during and/or after sliding the elongate tube member 820 distally along the guide catheter 802, toward a targeted vessel site. After decoupling the guide catheter 802 and elongate tube member 820, the tube member 820 may constrict, such that the longitudinal slit 829 closes or substantially closes, thereby narrowing the cross-sectional diameter of the tube member 820. In other embodiments, the slit 829 may not constrict upon decoupling, instead temporarily expanding only during insertion or removal of the guide catheter 802.

FIG. 8B illustrates a cross-sectional view of the guide catheter 800, taken along line 8-8 of FIG. 8A. While the longitudinal slit 829 is positioned above the guide catheter 802 in the configuration shown, the relative position of the slit 829 may vary. For example, the slit 829 may be positioned anywhere along the circumference of the elongate tube member 820, extending below or alongside the guide catheter 802, for instance. The width of the slit 829 may also vary. In embodiments, the width of the slit 829 in its unbiased, i.e., most narrow, configuration may be about 5%, 10%, 20%, 30%, 40%, 50% or more of the width of the guide catheter 802. The width of the slit 829 may depend on the diameter of the guide catheter 802 and/or the flexibility of the elongate tube member 802. For example, the width of the slit 829 may be smaller for a tube member 820 having greater flexibility, and vice versa.

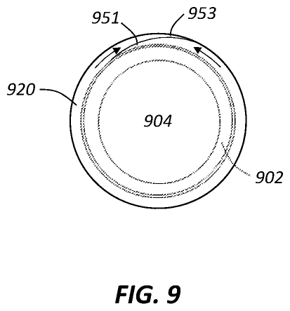

FIG. 9 illustrates a cross-sectional view of another elongate tube member 920 surrounding a guide catheter 902, which defines an inner lumen 904. The tube member 920 is biased to constrict around an outer surface of the guide catheter 902 via two overlapping ends 951, 953 configured to converge radially in the direction of the arrows. Upon extension of a portion of the tube member 920 beyond a distal end of the guide catheter 902, the ends 951, 953 may continue to slide past each other, further constricting the tube member 920 until its diameter is approximately equal to or less than the inner diameter of the guide catheter 902.

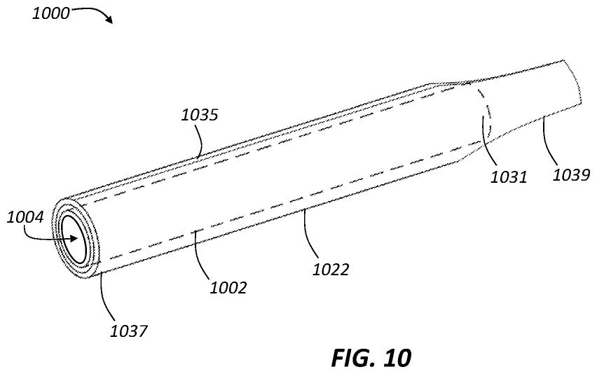

FIG. 10 illustrates a portion of a guide extension catheter 1000 surrounding a distal portion of a guide catheter 1002, which defines a lumen 1004. In this embodiment, the push member 1022 comprises an elongate tube defining a longitudinal slit 1035. Like the slit 829 illustrated in FIGS. 8A/8B, the slit 1035 can be defined by non-overlapping ends of the push member 1022, and can be formed to be resiliently closed, such that the slit 1035 can be forcibly peeled open to receive and/or remove the guide catheter 1002. The push member 1022 may be configured to constrict upon its extension beyond the guide catheter's distal end 1031. The entire length of the push member 1022 may be configured to constrict, or only a portion thereof. In some embodiments, only a distal portion 1039 may be configured to constrict, while a proximal portion 1037 may generally retain an approximately constant diameter. In such embodiments, the slit 1035 may be confined to the distal portion 1039 of the push member.

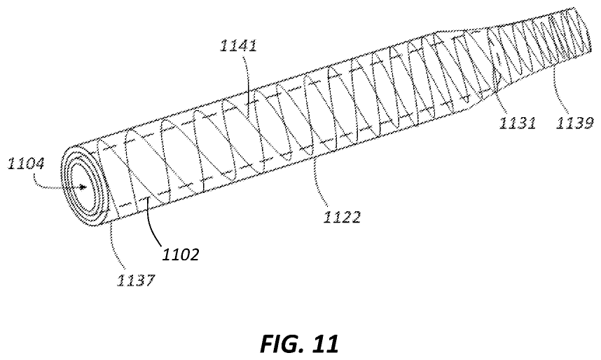

FIG. 11 illustrates a portion of another guide extension catheter 1100 surrounding a distal portion of a guide catheter 1102, which defines a lumen 1104. The push member 1122 again comprises an elongate tube configured to encompass and extend at least partially beyond a distal end 1131 of the guide catheter 1102. The push member 1122, or at least a distal portion 1139 thereof, may include an elastic material or structure configured to constrict, such as a shape-memory braid 1141, which may be comprised of nitinol. In some examples, the braid 1141 (or other constricting structure/material) may be confined to the distal portion 1139, such that only the distal portion is configured to constrict, e.g., upon extension beyond the guide catheter's distal end 1131. The length of the push member 1122 configured to constrict may vary, for example such that the constrictable portion spans from a distal end of the push member 1122 and constitutes approximately less than or equal to 5%, 10%, 20%, 30%, 40%, 50%, 60%, 70%, 80%, 90%, or 100% of the length of the push member 1122, or any length therebetween.

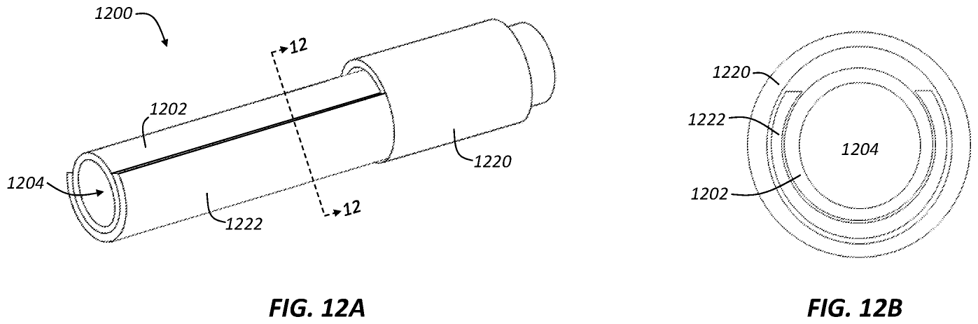

FIG. 12A illustrates a perspective view of another guide extension catheter 1200 comprised of an elongate tube member 1220 attached to a push member 1222. A portion of a guide catheter 1202 is shown extended through the elongate tube member 1220. In this embodiment, the push member 1222 comprises a concave track configured to couple with the guide catheter 1202 via a clipping or snapping mechanism. By reversibly coupling the push member 1222 with the guide catheter 1202 in this manner, the push member 1222 (and elongate tube member 1220) can be slidably guided to a vascular target site by the guide catheter 1202, thereby reducing the likelihood of the guide extension catheter 1200 buckling or twisting, or even perforating a vessel wall, upon receiving an axial force applied by an operating physician. As shown, the push member 1222 may extend around greater than half the circumference of the guide catheter 1202. The push member 1222 may comprise a relatively flexible, elastic or resilient material configured to bend upon coupling the guide catheter 1202 therewith. The push member 1222 and/or guide catheter 1202 may comprise a friction-reducing material, thereby allowing the two components to slide past each other during insertion of the guide extension catheter 1200.

FIG. 12B illustrates a cross-sectional view of the guide extension catheter 1200 taken along line 12-12 of FIG. 12A, showing the lumen 1204 defined by the guide catheter 1202, which is partially surrounded by the push member 1222 and completely surrounded at a distal portion by the elongate tube member 1220. In embodiments, the degree of enclosure defined by push member 1222 around the circumference of the guide catheter 1202 may vary, for example ranging from less than or equal to about 50%, 60%, 70%, 80%, 90%, or greater, or any degree therebetween.

Additional examples can include a push member having a shape that is approximately rectangular or flat, or a shape that changes along the length of the push member. The push member can also include multiple surfaces each having a distinct shape or curvature, for example as described in U.S. patent application Ser. No. 15/581,176.

In some embodiments, the push member can be an elongated solid wire of constant or varying dimensions and can be made of a polymeric or metallic material, such as high tensile stainless steel (e.g., 304V, 304L or 316LV), mild steel, nickel-titanium allows, nickel-chromium-molybdenum alloys, nickel-copper alloys, nickel-tungsten alloys or tungsten alloys. The push member can be coated with a hydrophilic, silicone or other friction-reducing material. The stiffness of the push member may be uniform, or substantially uniform, along its length, or may define areas having variable stiffness along its length. For example, the push member may be more flexible near its distal end than its proximal end.

FIG. 13A illustrates an example reinforcement member 1300, which may be included in some embodiments to increase the stiffness of the elongate tube member 1302 of a guide extension catheter (only a portion of which is shown). As described above, the reinforcement member 1300 may be sandwiched between two polymer layers constituting the elongate tube member 1302. The reinforcement member 1300 can include a plurality of longitudinal bars or strips 1304, which may be interlaced with one or more cross-bars or strips 1306. The strips 1304, 1306 may be arranged perpendicularly, or substantially perpendicularly, with respect to each other, or they may be diagonally arranged. In some examples, only the longitudinal or the cross strips may be included. The reinforcement member 1300 can extend around the entire perimeter of the elongate tube member 1302, or only a portion thereof.

FIG. 13B illustrates another example reinforcement member 1308 included with an elongate tube member 1310 (only a portion of which is shown). In this example, the reinforcement member 1308 can be comprised of spiraling bars or strips 1312, 1314, which may criss-cross. In embodiments, strips in only one spiral direction, i.e., 1312 or 1314, may be included. Any suitable angle or combination of angles of the spiral with respect to the longitudinal axis of the tube may be used. Like reinforcement member 1300, reinforcement member 1308 can be sandwiched between individual layers constituting the elongate tube member 1310. The particular configuration of the reinforcement member, its location and/or length may vary in different embodiments of the guide extension catheters disclosed herein, which are not confined to examples including reinforcement members, or specific embodiments thereof. The materials constituting the reinforcement member may also vary. In examples, the reinforcement member can include stainless steel, a platinum alloy, and/or one or more polymers, for instance.

### EXAMPLES

The above Detailed Description is intended to be illustrative and not restrictive. The above-described embodiments (or one or more features or components thereof) can be used in varying combinations with each other unless clearly stated to the contrary. Other embodiments can be used, such as by one of ordinary skill in the art upon reviewing the above Detailed Description. Also, various features or components have been grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples, with each example standing on its own as a separate embodiment.

### Closing Notes

The above Detailed Description includes references to the accompanying drawings, which form a part of the Detailed Description. The Detailed Description should be read with reference to the drawings. The drawings show, by way of illustration, specific embodiments in which the present guide extension catheters and related methods can be practiced. These embodiments are also referred to herein as “examples.”

Certain terms are used throughout this patent document to refer to particular features or components. As one skilled in the art will appreciate, different people may refer to the same feature or component by different names This patent document does not intend to distinguish between components or features that differ in name but not in function. For the following defined terms, certain definitions shall be applied unless a different definition is given elsewhere in this patent document. The terms “a,” “an,” and “the” are used to include one or more than one, independent of any other instances or usages of “at least one” or “one or more.” The term “or” is used to refer to a nonexclusive or, such that “A or B” includes “A but not B,” “B but not A,” and “A and B.” All numeric values are assumed to be modified by the term “about,” whether or not explicitly indicated. The term “about” refers to a range of numbers that one of skill in the art considers equivalent to the recited value (i.e., having the same function or result). In many instances, the term “about” can include numbers that are rounded to the nearest significant figure. The recitation of numerical ranges by endpoints includes all numbers and sub-ranges within and bounding that range (e.g., 1 to 4 includes 1, 1.5, 1.75, 2, 2.3, 2.6, 2.9, etc. and 1 to 1.5, 1 to 2, 1 to 3, 2 to 3.5, 2 to 4, 3 to 4, etc.). The terms “patient” and “subject” are intended to include mammals, such as for human or veterinary applications. The terms “distal” and “proximal” are used to refer to a position or direction relative to an operating physician. “Distal” and “distally” refer to a position that is distant from, or in a direction away from, the physician. “Proximal” and “proximally” refer to a position that is near, or in a direction toward, the physician. And the term “interventional device(s)” is used to include, but is not limited to, balloon catheters, stents and stent catheters.

The scope of the present guide extension catheters and methods should be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended; that is, a device or method that includes features or components in addition to those listed after such a term in a claim are still deemed to fall within the scope of that claim. Moreover, in the following claims, the terms “first,” “second” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

What is claimed is:

1. A guide extension catheter for use with a predefined length guide catheter, the guide extension catheter comprising:
an elongate tube member defining a lumen having a cross-sectional inner diameter through which the guide catheter is insertable; and
a push member that is rigid enough to allow the elongate tube member to be urged over the guide catheter, the push member being proximal of, operably connected to, and more rigid along a longitudinal axis than the elongate tube member, the push member having a maximal cross-sectional dimension that is smaller than a cross-sectional outer diameter of the elongate tube member and having a length such that when combined with a length of the elongate tube member, forming a guide extension catheter length along the longitudinal axis that is longer than the guide catheter.

2. The guide extension catheter of claim 1, further comprising an introducer sheath defining a lumen having a cross-sectional inner diameter through which the elongate tube member is insertable.

3. The guide extension catheter of claim 1, wherein the elongate tube member includes a longitudinal slit along a length thereof, the longitudinal slit biased toward a closed position.

4. The guide extension catheter of claim 1, wherein the cross-sectional inner diameter of the elongate tube member is configured to adjust in response to a force applied thereto.

5. The guide extension catheter of claim 4, wherein the elongate tube member comprises a shape memory braid.

6. (canceled)

7. The guide extension catheter of claim 4, wherein the cross-sectional inner diameter of the elongate tube member is configured to decrease until the cross-sectional inner diameter is approximately equal to a cross-sectional outer diameter of the guide catheter.

8. (canceled)

9. The guide extension catheter of claim 1, wherein the push member comprises a rod or a wire.

10. The guide extension catheter of claim 1, wherein the push member comprises a longitudinal groove along the length thereof.

11. The guide extension catheter of claim 1, wherein the elongate tube member comprises an inner polymer layer and an outer polymer layer.

12. The guide extension catheter of claim 1, wherein the elongate tube member, when its distal end is extended distally of a distal end of the guide catheter, provides support for delivery of an interventional cardiology device in the form of a stent, a stent catheter, or a balloon catheter.

13. A method, comprising:
advancing a distal end of a guide catheter having a continuous lumen through a blood vessel to an ostium of a coronary artery;
advancing a distal end of a guide extension catheter beyond the distal end of the guide catheter, including advancing a distal end portion of a tubular structure of the guide extension catheter over and beyond the distal end of the guide catheter while a proximal end portion of the tubular structure remains around the guide catheter, the tubular structure having a cross-sectional inner diameter sized to be positioned around the guide catheter, the guide catheter defining a lumen having a cross-sectional inner diameter through which interventional cardiology devices are insertable; and
further advancing a push member of the guide extension catheter that is proximal of, operably connected to, and more rigid along a longitudinal axis than the tubular structure, along an outer surface of the guide catheter, the push member having a maximal cross-sectional dimension that is smaller than a cross-sectional outer diameter of the tubular structure and having a length such that when combined with a length of the tubular member, forming a guide extension catheter length that is longer than the guide catheter.

14. The method of claim 13, further comprising inserting an introducer sheath into an entry point of a patient, the introducer sheath defining a lumen having a cross-sectional inner diameter through which the tubular structure is insertable.

15. The method of claim 13, wherein the tubular structure includes a longitudinal slit along a length thereof, the longitudinal slit biased toward a closed position.

16. The method of claim 15, further comprising inserting the guide catheter into the tubular structure by urging the guide catheter through the longitudinal slit prior to advancing the distal end of the guide extension catheter over the guide catheter.

17. The method of claim 16, further comprising removing the guide catheter from the tubular structure by urging the guide catheter through the longitudinal slit.

18. The method of claim 13, wherein the push member comprises a rod, a wire, or a rail structure without a lumen.

19. The method of claim 13, wherein the cross-sectional inner diameter of the tubular structure is configured to adjust in response to a force applied thereto.

20. The method of claim 19, wherein the tubular structure comprises a shape memory braid comprised of nitinol.

21. The guide extension catheter of claim 3, wherein the elongate tube member is biased to wrap around an outer surface of the guide catheter by way of its two longitudinal overlapping ends.

22. The guide extension catheter of claim 1, wherein at least a distal portion of the elongate tube member, when extended distally of a distal end of the guide catheter, is configured to decrease in cross-sectional diameter.
